(19) United States
(12) Patent Application Publication
(10) Pub. No.: US 2008/0124395 A1
(43) Pub. Date: May 29, 2008

(54) FORMULATIONS AND DEVICES FOR TREATMENT OR PREVENTION OF NEURAL ISCHEMIC DAMAGE

(76) Inventors: Weiliam Chen, Mount Sinai, NY (US); John M. Abrahams, Scarsdale, NY (US); Renato Rozental, Elmsform, NY (US)

Correspondence Address: SCHWEGMAN, LUNDBERG & WOESSNER, P.A., P.O. BOX 2938, MINNEAPOLIS, MN, 55402, US

(21) Appl. No.: 11/425,958

(22) Filed: Jun. 22, 2006

(51) Int. Cl. A61K 9/00; A61M 31/00; A61P 35/00; A61K 47/36; A61K 47/32; A61P 25/00

(52) U.S. Cl. 424/486; 604/506; 514/179; 514/772.6; 424/488; 424/422

(57) ABSTRACT

A formulation is provided for treatment or prevention of neural ischemic damage, or for treatment of stroke, hemorrhage, trauma, epilepsy, tumor, or any disease of the brain, the formulation comprising a mixture of a substantially solid, substantially water-insoluble, biocompatible, polymeric material, and a gap junction inhibitor such as carbenoxolone. The polymeric material may comprise a synthetic polymer such as EVA, for example in the physical form of a fiber, or a natural polymer such as a chitosan derivative, for example in the physical form of a hydrogel, disposed on or in a medical device such as a stent, which is implanted within the tissue of the patient.

### FIELD OF THE INVENTION

The invention relates to a formulation of a gap junction inhibitor such as carbenoxolone in a polymeric material and to a medical device comprising the formulation for treatment or prevention of neural ischemic damage as from stroke.

### BACKGROUND

Every year in the United States about 600,000 people suffer a stroke, and for about 160,000 of these the stroke is fatal.

Approximately three quarters of all stroke cases involve ischemic stroke. The remaining quarter are hemorrhagic. Ischemic stroke most commonly results from thromboembolic occlusion of blood vessels in the brain of a diameter greater than 1 mm. The interruption of the flow of oxygenated blood to the neural tissues of the brain causes pathological alterations in the tissues that may be reversible if oxygenation is restored within about 2 hours, but become irreversible with a greater lapse of time. The severity of the damage caused by a stroke depends on both the degree and the duration of the ischemia.

The region immediately proximal to the occlusion that are usually supplied with oxygen from those capillaries that are blocked by the stroke is referred to as the ischemic focus or core, and it is typically the site of the most immediate, severe damage, but areas surrounding the ischemic focus are also at risk. These surrounding areas, known as the “ischemic penumbra,” undergo changes as the ischemic condition persists that can become irreversible, increasing the severity of the neural deficit that results from a stroke. Thus, an indicated therapy for patients afflicted with stroke includes protection and treatment of cells within the ischemic penumbra, so that affected cells are treated and yet-unaffected cells are protected from damage. Inflammation may be a secondary consequence of ischemic stroke that causes further damage to neural tissue. Excess glutamate, inflammatory cytokines, and other effects of inflammation can result in further damage through apoptosis.

Additional damage to neural tissue may occur as a result of the sudden removal of the arterial blockage that caused the stroke. Known as “reperfusion injury,” the shock of re-oxygenation of the ischemic tissue, including that tissue in the ischemic penumbra, can accentuate the permanent damage that results from the blockage.

The compound carbenoxolone (CBX) has been shown to be beneficial in the treatment or prevention of ischemic damage, as has been described in published U.S. Patent Application US 2004/0058852, incorporated herein by reference, where a method is provided for prevention of hypoxic-ischemic damage to newborn infants using CBX, administered either before or after delivery. Treatment of traumatic brain injury is also described.

It is believed that the beneficial effects seen with the use of CBX in this therapy are attributable to the property the compound possesses as a gap junction inhibitor. A gap junction inhibitor (GJI) is a molecular entity that interferes with the formation or functioning of intercellular gap junction channels. Gap junction channels are formed between adjacent cells, and allow for the direct exchange of small molecules including salts and organic substances between the cells. They are widespread throughout normal healthy tissue, being found in substantially all tissue types except striated muscle and non-nucleated cells such as erythrocytes and platelets. The small-molecule exchange that the channels enable is not mediated by transmembrane transport or by receptor-mediated endocytosis, but takes place by direct diffusion of the substances through the gap junction channel. Gap junction channels are believed to be formed by cells through the expression and action of proteins termed connexins. Connexins are membrane-spanning proteins that aggregate into macromolecular complexes termed connexons, typically involving six connexin molecules, that are able to fuse with connexons on adjacent cells to form the intercellular gap junction channel. Once the channel is formed, some substances that are dissolved in the cytosol can freely diffuse from cell to cell. Gap junctions are known to provide a fundamental mechanism of communication between many cell types in the body. See, for example, W. H. Evans and S. Boitano (2001), “Connexin mimetic peptides: specific inhibitors of gap-junctional intercellular communication,” Biochemical Society Transaction, 29(4), 606-612.

It is believed that ischemic cells produce toxic substances that may pass into adjacent cells through the gap junction channels. This effect is believed to be responsible, at least in part, for the phenomenon of the ischemic penumbra in stroke. Cells that have been directly deprived of oxygen through interruption of blood flow act as sources for toxic materials that damage adjacent cells linked to them through gap junction channels. Thus, blockage of the gap junction channels could serve to prevent the passage of these toxic substances into the adjacent cells, reducing the potential damage to those adjacent cells, and thus reducing the size of the ischemic penumbra that can form around an ischemic core. Efforts have been made to identify molecular entities that can serve to close gap junction channels or to prevent their formation or their functioning, with the rationale that such entities could be effective in mitigating the effects of a stroke. For example, see David C. Spray, Renato Rozental and Miduturu Srinivas (2002), “Prospects for Rational Development of Pharmacological Gap Junction Channel Blockers,” Current Drug Targets, 3, 455-464. See also “How to Close a Gap Junction Channel: Efficacies and Potencies of Uncoupling Agents,” Renato Rozental, Miduturu Srinivas and David C. Spray, Methods in Molecular Biology, v. 154, chap. 25, ed R. Bruzzone and C. Giaume, Humana Press, Tolowa, N.J.

Gap junction inhibitors include, besides CBX, a number of structurally diverse compounds including 18α- and 18β-glycerrhetinic acid, connexin external loop analogs such as the peptides GAP-27 (SRPTEKTIFII) and GAP-26 (VCYDKSFPISHVR), antibodies against external loop domains of connexins, fatty acids and their derivatives such as arachidonic acid, oleic acid, oleamide and anandamide, lipophilic compounds such as halothane, octanol, and drugs including flufenamic acid and niflumic acid. Such compounds offer promise in the treatment of malconditions where intercellular diffusion of toxic materials plays a role in the spread of ischemia from cell to cell.

### SUMMARY OF THE INVENTION

The present invention comprises a formulation that includes a gap junction inhibitor (GJI) such as carbenoxolone, and an organic polymeric material. A mass of the polymeric material, which may comprise either a synthetic polymer or a natural polymer, contains the GJI dispersed within. The formulation may be a solid that can be shaped into a fiber or a film, or may form a hydrogel through gelation of a premix that can be emplaced within living tissue. A polymer of the invention is substantially solid and substantially water insoluble at the time of emplacement within the tissue of a patient in need thereof. By substantially solid is meant that the polymer, after emplacement, does not flow but remains as a coherent mass. By substantially water-insoluble is meant that the polymer does not immediately or rapidly dissolve in water or body fluids with a time course of minutes or hours. The polymer can be biodegradable, meaning that it can disintegrate and dissolve into components over the time course of days, weeks or months.

The polymer serves to retain the GJI in the vicinity of the position of emplacement of the polymeric mass, allowing selective delivery of the GJI to a defined region of the neural tissue, such as an ischemic core or an ischemic penumbra resulting from a stroke, in a controlled manner over a period of time. This type of medicament is useful for treatment of or prevention of ischemic damage in neural tissue, such as occurs in human patients afflicted with stroke or who have received a traumatic injury to the brain.

The invention further provides a medical device, for example a stent, on or within which the polymeric material containing the GJI is disposed. The polymeric material with the contained GJI can be a hydrogel, which can be coated onto the stent or a premix for which can be pumped into a void within a hollow, porous stent wire adapted to receive it, wherein it solidifies. Or, the polymeric material with the contained GJI can be dissolved in a suitable solvent and applied to the stent, such as by electrospray. Alternatively, the polymeric material with the contained GJI can be in the physical form of a fiber or a film, which is mechanically disposed on or within the medical device. For example, a GJI-containing polymer fiber may be wrapped around a stent or woven into a stent.

The invention further comprises methods of use of the formulation, and of medical devices in or on which the formulation is disposed, in the prevention and treatment of neural ischemic damage as a result of stroke, traumatic brain injury, and other causes. The formulation can be emplaced directly at a desired location, such as near the site of an ischemic focus with the brain, for example using a cannula. Alternatively, a medical device comprising the formulation may be inserted into tissue, for example into a blood vessel, at or near the site of an ischemic focus within the brain.

A process is further provided for the manufacture of a medical device for treatment of neural ischemic damage, such as occurs from stroke. The medical device, for example a stent, comprises the polymer containing the GJI agent. The formulation of the invention comprising the medicament and the polymer is applied to the medical device, for example, by dipping the device in a solution of the formulation, or spraying the device with the formulation, or pumping the formulation into a void in the device adapted to receive it, or wrapping of the device with a thread or a film formed from the polymer/GJI formulation.

### BRIEF DESCRIPTION OF THE DRAWINGS

FIG. 1 shows an embodiment of a stent comprising a formulation of the present invention.

### DETAILED DESCRIPTION OF PREFERRED EMBODIMENTS

### Definitions

As used herein, the terms “treat” “treating,” “treatment,” refer to medical procedures that attempt to lessen the severity or reverse the effects of a malcondition, such as neural ischemic damage, such that the pathological results that would otherwise have resulted from the malcondition are less likely to occur. The terms as used herein refer to inhibiting the disorder or disease, for example, arresting the development of the disorder or disease; relieving the disorder or disease, for example, causing regression of the disorder or disease; or relieving the condition caused by the disease or disorder, for example, stopping the symptoms of the disease or disorder. For example, a procedure that reverses ischemic damage that has occurred to neurons with the ischemic penumbra is a treatment within the meaning herein.

The terms “prevent,” “preventative,” “prevention,” “protect,” and “protection” refer to medical procedures that keep the malcondition from occurring in the first place. The terms mean that there is no or a lessened development of disease or disorder where none had previously occurred, or no further disorder or disease development if there had already been development of the disorder or disease. For example, a procedure that blocks the occurrence of ischemic damage in neurons that would otherwise be expected to be within the ischemic penumbra is a preventative procedure.

“Treatment” of neural ischemic damage is carried out with the goal of causing healing of damaged or compromised neurons to restore normal functions, whereas “prevention” of neural ischemic damage is carried out with the intention of blocking or avoiding damage to otherwise healthy neurons.

The terms “therapy,” and “therapeutic” refer to either “treatment” or “prevention,” thus, agents that either treat damage or prevent damage are “therapeutic.”

“Therapeutic agents,” as the term is used herein, comprise pharmaceuticals, bioactive natural products, enzymes, antibodies, peptides and peptidomimetics, antisense nucleotides or their analogs, or any other such agents as are medically advantageous for use to treat neural ischemia through interference with the formation or functioning of gap junction channels.

A “controlled release formulation” is a formulation of a therapeutic agent wherein the release of the agent into the living body tissue of a patient is intended or designed to take place over a period of time, reducing the necessity for repeated dosing of the agent in the course of therapy for a malcondition comprising ischemia or damage to central nervous system tissue.

“Neural” as used herein means of or pertaining to the nervous system, including neurons as well as accessory cell types and tissues, for example neuroglia or myelin sheaths.

“Ischemia” and “ischemic” refer to a state of oxygen-deficiency in tissue, for example in central nervous system tissue, resulting from an insufficiency of oxygenated hemoglobin reaching the affected cells or areas, such as occurs in stroke.

“Stroke” refers to a malcondition affecting the brain wherein a blood vessel supplying brain tissue either becomes blocked (“ischemic stroke”) or ruptures (“hemorrhagic stroke”). The interruption of neural functions and the death of neural cells can produce stroke symptoms including mental disability and death.

“Gap junctions” and “gap junction channels” are intercellular channels or pores as described above wherein membrane-spanning structures on adjacent cells form and couple, creating a direct cell-to-cell connection. A “gap junction inhibitor” is a molecular entity that interferes with the formation or functioning of intercellular gap junction channels or hemichannels. An example of a gap junction inhibitor is carbenoxolone. Another example is the peptide GAP 27 (SRPTEKTIFII). Yet another example is an antibody specific for the extracellular connexon domain.

When it is stated that a formulation of the invention is implanted “in proximity to” an ischemic focus, what is meant is that the formulation is implanted in a spatial relationship to the site of the oxygen-deficient tissue such that the gap junction inhibitor that is released from the formulation can achieve a therapeutically functional concentration in and around cells that are at risk for damage from the stroke or other malcondition that induced the ischemia.

The term “ethylene vinyl acetate copolymer” refers to the polymer also known as EVA wherein the monomers ethylene and vinyl acetate are copolymerized via their double bonds. The monomeric units can be present in a range of relative concentrations and the molecular weight of the polymeric product can range from a few thousand into the hundreds of thousands of daltons.

The term “poly(methyl methacrylate)” refers to the material obtained by polymerization of methyl methacrylate. The molecular weight of this polymer can likewise range from a few thousand into the hundreds of thousands of daltons.

As used herein, “chitosan” refers to an amino-polysaccharide polymer, either obtained from a natural source such as chitin, or synthetically prepared. Chitosan is predominantly a polymer of β-1,4-linked 2-amino-2-deoxyglucose units. When prepared from a natural source, the usual natural source is chitin, a major constituent of the shells of crabs, shrimp and other arthropods and of fungal cell walls. Chitosan is prepared from deacetylation of chitin. A sample of chitosan typically comprises polymeric chains of various lengths. A “chitosan derivative” is a polymer structurally related to chitosan that can be obtained by chemical or biochemical modification of chitosan. An example of a chitosan derivative is an alkylated chitosan.

As the term is used herein, an “alkylated chitosan” is a material composed of chitosan molecules to which carbon-containing molecules have been covalently bonded. For example, methylation of chitosan, in which bonds are formed between methyl radicals or groups and atoms within the chitosan molecule, such as nitrogen, oxygen or carbon atoms, provides an alkylated chitosan within the definition used herein. Other carbon-containing groups may likewise be chemically bonded to chitosan molecules to produce an alkylated chitosan. An example is acrylated chitosan. Another example is PEG-chitosan, a type of poly(oxyalkylene)chitosan.

A “poly(oxyalkylene)chitosan” is an alkylated chitosan as defined herein. A “poly(oxyalkylene)” group is a polymeric chain of atoms containing a monomeric repeating unit wherein two carbon atoms of an alkylene group are bonded to oxygen atoms. The carbon atoms of the alkylene group repeating unit may themselves bear additional radicals. For example, if the alkylene group is ethylene, and the ethylene groups are unsubstituted, the poly(oxyalkylene) is a poly(oxyethylene). If each ethylene group bears a single methyl group, the resulting poly(oxyalkylene) group is a poly(oxy-1,2-propylene). If a three-carbon linear alkylene group is disposed between the oxygen atoms, the entity is a poly(oxy-1,3-propylene)

A poly(oxyalkylene) such as poly(oxyethylene) may be of a wide range of lengths, degrees of polymerization, and therefore molecular weights.

Poly(oxyethylene) is of a general molecular formula of the structure [—CH₂—CH₂—O—CH₂—CH₂—O—]ₙ, where n may range from about 3 upwards to 10,000 or more. Often referred to as “polyethyleneglycol” or “PEG” derivatives, these polymeric chains are of a hydrophilic, or water-soluble, nature.

Thus, a poly(oxyalkylene)chitosan is a chitosan derivative to which poly(oxyalkylene) groups are covalently attached. A carbon atom or an oxygen atom of the poly(oxyalkylene) group forms a covalent bond with an atom of the chitosan chain. Typically, a carbon atom of the poly(oxyalkylene) group forms a covalent bond with a chitosan nitrogen atom, although bonds to oxygen or even carbon atoms of the chitosan chain may exist. Poly(oxyethylene)chitosan is often referred to as “polyethyleneglycol-grafted chitosan” or “PEG-g-chitosan” or “PEG-chitosan.” Preferably, the degree of cross-linking of the chitosan chains by the poly(oxyalkylene) groups is minimal; therefore only one end of a poly(oxyalkylene) group is typically bonded to a chitosan chain.

The end of the poly(oxyethylene) chain that is not bonded to the chitosan chain may be a free hydroxyl group, or may comprise a capping group such as methyl or acetate. Thus, “polyethylene glycol” or “poly(oxyethylene)” or “poly(oxyalkylene)” as used herein includes polymers of this class wherein one, but not both, of the terminal hydroxyl groups is capped, such as with a alkyl or acyl group. In a preferred method of preparation of the poly(oxyethylene)chitosan, use of a polyethyleneglycol capped at one end, such as MPEG (methyl polyethyleneglycol) may be advantageous in that if the PEG is first oxidized to provide a terminal aldehyde group, which is then used to alkylate the chitosan via a reductive amination method, blocking of one end of the PEG assures that no difunctional PEG that may crosslink two independent chitosan chains is present in the alkylation reaction. It is preferred to avoid crosslinking in preparation of the poly(oxyethylene)chitosan of the present invention.

An alkylated chitosan can also be a chitosan to which other carbon-containing moieties are linked. An “acrylated chitosan” as the term is used herein is an alkylated chitosan wherein acrylates have been allowed to react with, and form chemical bonds to, the chitosan molecule. An acrylate is a molecule containing an α,β-unsaturated carboxyl group; thus, acrylic acid is prop-2-enoic acid. An acrylated chitosan is a chitosan wherein a reaction with acrylates has taken place. The acrylate can bond to the chitosan by any possible configuration of bonds, although a Michael addition of the chitosan nitrogen atom with the acrylate is believed to be the major mode of coupling.

As used herein, a “hydrogel” refers to a material of solid or semi-solid texture that comprises water. Hydrogels are formed by a three-dimensional network of molecular structure within which water, among other substances, may be held. The three-dimensional molecular network may be held together by covalent chemical bonds, or by ionic bonds, or by any combination thereof. A common example of a hydrogel is gelatin, a protein, that “sets up” or forms a gel from a sol upon heating and subsequent cooling. As used herein, a “sol” is a substantially liquid composition that can set up to form a gel under certain conditions. Polysaccharides such as starches may also form hydrogels. Still other hydrogels may be formed from a mixture of two or more materials that undergo chemical reaction with each other to create the three-dimensional molecular network that provides the hydrogel with a degree of dimensional stability. A mixture of materials that interact or react with each other to form a hydrogel are referred to herein as a “premix.” Thus, a “premix” refers to a mixture of materials that after mixing form a sol that will gel, or set up, to form the hydrogel. A premix is of a liquid or semi-liquid texture such that it can be pumped or transferred by the methods usually used for liquids, such as flow through tubes, but after gelation occurs, the resulting hydrogel is substantially solid such that it is not readily pumped and does not readily flow.

The act of “gelation” refers to the formation of a gel from a sol. In some cases, the sol may consist of a single material dispersed in a solvent, typically water. In other cases, the sol may consist of more than a single material dispersed in a solvent wherein the several materials will eventually react with each other to form a gel, and when the solvent in which they are dispersed comprises water, the gel is a hydrogel. The hydrogels claimed herein are of the type that are formed by the mixture of more than a single component.

An “acidic polysaccharide” is a polymer comprising carbohydrate moieties wherein the polymer also comprises carboxylic acid groups. An example of an acidic polysaccharide is hyaluronic acid.

An “oxidized polysaccharide” is a polymer comprising carbohydrate moieties wherein the polymer also comprises aldehyde groups. An example of an oxidized polysaccharide is oxidized dextran.

When referring to the “molecular weight” of a polymeric species such as an alkylated chitosan, a weight-average molecular weight is being referred to herein, as is well known in the art.

A “degree of substitution” of a polymeric species refers to the ratio of the average number of substituent groups, for example an alkyl substituent, per monomeric unit of the polymer as defined.

A “degree of polymerization” of a polymeric species refers to the number of monomeric units in a given polymer molecule, or the average of such numbers for a set of polymer molecules.

As used herein, a “polybasic carboxylic acid” means a carboxylic acid with more than one ionizable carboxylate residue per molecule. The carboxylic acid may be in an ionized or salt form within the meaning of the term herein. A dibasic carboxylic acid is a polybasic carboxylic acid within the meaning herein. Thus, adipic acid is a polybasic carboxylic acid, having two ionizable carboxylate residues per molecule. Disodium adipate is a polybasic carboxylic acid within the meaning of the term herein. Alternatively, the polybasic carboxylic acid may have hundreds or thousands of ionizable carboxylate groups per molecule; for example, hyaluronan, also known as hyaluronic acid, is a polybasic carboxylic acid within the meaning assigned herein. The hyaluronan or hyaluronic acid may be in an ionized or salt form within the meaning used herein. Thus, sodium hyaluronate is a polybasic carboxylic acid within the meaning of the term as used herein. Another example of a polybasic carboxylic acid is the semi-synthetic polymer carboxymethylcellulose. Yet another example of a polybasic carboxylic acid is oxidized hyaluronic acid.

A “dehydrating reagent” as used herein refers to a molecular species that takes up the elements of water from a reaction, serving to drive a coupled reaction due to thermodynamic factors. A dehydrating reagent is an compound that undergoes reaction of covalent bonds upon taking up the elements of water, as opposed to merely absorbing water into physical particles or the like. Preferably a dehydrating reagent is an organic compound. A specific example of a dehydrating reagent is a carbodiimide, that takes up the elements of water and undergoes changes in covalent bonds to ultimately yield a urea derivative.

As used herein, a “carbodiimide” is a class of organic substances comprising a R—N═C═N—R′ moiety. Any organic radicals may comprise the R and R′ groups. A water-soluble carbodiimide is a carbodiimide that has sufficient solubility in water to form a homogeneous solution at concentrations suitable to carry out the gelation reaction as described herein. The water-soluble diimide EDCI is 1-ethyl-3-N,N-dimethylaminopropylcarbodiimide.

A “carboxyl activating reagent” as used herein refers to a molecular species that interacts with a carboxyl group in such a way as to render the carbonyl of the carboxyl group more susceptible to nucleophilic attack, as by an amine to yield an amide. This activation may take place by formation of a complex or by formation of a covalent intermediate. A specific example of a carboxyl activating reagent is an N-hydroxy compound that can form an N-hydroxy ester of the carboxylic acid group, increasing the reactivity of the carbonyl moiety to nucleophilic addition of a molecular species such as an amine.

The term “N-hydroxy compound” refers to an organic compound comprising a chemical bond between a hydroxyl group and a nitrogen atom. Preferred N-hydroxy compounds such as N-hydroxysuccinimide and N-hydroxybenztriazole (1-hydroxy benzotriazole) are well known in the art as reagents that form esters with carboxylic acid groups and serve to activate the carboxylic acid group in reactions with nucleophiles.

Carbenoxolone is a pentacyclic triterpene derivative of the formula:

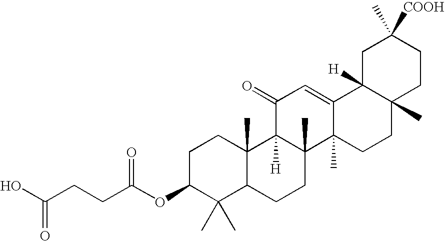

Beta-glycyrrhetinic acid is carbenoxolone without the hemisuccinate ester on O-3. Alpha-glycyrrhetinic acid is the epimer of beta-glycyrrhetinic acid at C-18, the hydrogen at the D/E ring junction.

A “stent” is a medical device used to support a duct or vessel, such as a blood vessel, that is typically disposed on the interior of the vessel such that fluid flow is maintained through the vessel. A stent is formed from a biocompatible material, which may be a metal such as platinum, stainless steel, or nitinol. A stent can be formed from a “memory metal” which is a metal structure that can be compressed or deformed under pressure but readily resumes its original shape when the pressure is released. An example of a memory metal is nitinol. A stent can assume a variety of physical configurations forming a tubular structure, including a helix, two intertwined helices, or a mesh.

The “target tissue” is the tissue into which it is desired to infuse the gap junction inhibitor, such as brain tissue adjacent to an ischemic focus as is caused by a stroke, where the therapeutic effect of the gap junction inhibitor is desired to be exerted. The target tissue may be the tissue suffering from damage, as at the immediate site of the stroke, or may be adjacent tissue that is desired to be protected from damage.

### DETAILED DESCRIPTION

A gap junction inhibitor for use in the formulation of the invention includes any molecular entity known to interfere with the formation or functioning of intercellular gap junctions. For example, known gap junction inhibitors include: the triterpene derivatives carbenoxolone, 18α- and 18β-glycerrhetinic acids; connexin external loop analogs such as the peptides GAP-27 (SRPTEKTIFII) and GAP-26 (VCYDKSFPISHVR); antibodies against external loop domains of connexins; fatty acids and their derivatives such as arachidonic acid, oleic acid, oleamide and anandamide; other lipophilic compounds such as halothane and octanol; flufenamic acid, and niflumic acid. The use of carbenoxolone and other gap junction inhibitors in therapeutic applications against ischemic damage in neonates and resulting from traumatic brain injury is described, for example, in U.S. Patent Application Publication No. 2004/0058852, which is incorporated herein by reference.

A polymer for use in the formulation of the invention may be a synthetic polymer, for example, ethylene-vinyl acetate copolymer (EVA) or methyl methacrylate (MMA), or a combination thereof. A polymer of the invention may form a hydrogel in water, optionally in conjunction with other constituents. A hydrogel of the invention can be formed at least in part from a naturally occurring polymer or a derivative thereof, for example, a chitosan derivative. In any case, the formulation is a solid or semi-solid material when disposed within the living tissue, and retains its physical integrity upon exposure to an aqueous medium for at least a period of time over which the gap junction inhibitor (GJI), such as carbenoxolone, continues to be released into surrounding tissue. The formulation of the invention releases the GJI over a period of time, which is advantageous in treatment or prevention of a persistent or ongoing condition. The formulation of the invention is biocompatible, and can be biodegradable.

In one embodiment according to the present invention, a hydrogel comprising the GJI is formed by gelation of a premix comprising an organic polymer in an at least partially aqueous medium. The organic polymer can comprise chitosan or a chitosan derivative and the premix can be formed by addition of a polybasic carboxylic acid in an aqueous medium or by addition of an oxidized carbohydrate in an aqueous medium. The premix may comprise additional components, for example a dehydrating reagent or a carboxyl activating reagent. The premix, after the mixing together of the ingredients, expeditiously forms a hydrogel that is substantially non-liquid and insoluble in aqueous media. Prior to gelation, the premix is substantially liquid for a relatively short period of time and may be disposed onto a medical device such as a stent that is then implanted into the patient after formation of the hydrogel. Alternatively, the premix may be directly emplaced within tissue, such as via a cannula, where it gels in situ to form the hydrogel containing the GJI. The hydrogel is preferably biocompatible and biodegradable, allowing for its eventual absorption by living tissues following the release of the medicament. The period of time required for gelation is suitable for the particular application in which the premix is used.

In the embodiment comprising a hydrogel, the GJI can be introduced into the premix when the premix is prepared. The GJI is preferably substantially water-soluble at a pH around 7.0-7.4 at a suitable concentration, and thus is dissolved directly in the premix, but the GJI may first be dissolved in a suitable solvent, for example DMSO, NMP, or ethanol, prior to addition to the premix, such that the GJI is homogeneously dispersed within the premix prior to formation of the hydrogel.

For example, a hydrogel comprising an alkylated chitosan and a polybasic carboxylic acid may be formed from a premix comprising these substances, as is disclosed in U.S. patent application Ser. No. 11/379,182, filed Apr. 18, 2006, which is incorporated herein by reference. However other hydrogel formulations may be employed without departing from the principles of the invention.

The premix sol and the resulting hydrogel that forms from the sol prepared from the alkylated chitosan and the polybasic carboxylic acid are suitable for contact with living biological tissue, being biocompatible and preferably biodegradable. Thus, a hydrogel of the invention can remain in contact with living biological tissue within a human patient for an extended period of time without damaging the tissue on which it is disposed. Eventually, the hydrogel preferably biodegrades into soluble, non-toxic components which are removed from the site by the circulatory system.

A preferred embodiment of a premix that forms a hydrogel according to the present invention comprises a poly(oxyethylene)chitosan. A preferred degree of substitution for a poly(oxyethylene)chitosan is about 0.35 to about 0.95. A particularly preferred degree of substitution is about 0.5.

It should be understood that other poly(oxyalkylene) groups may be substituted for the poly(oxyethylene) group. For example, a poly(oxypropylene)chitosan may be used in place of, or in addition to, the poly(oxyethylene)chitosan. The molecular weight of a chitosan according to the present invention may vary widely without departing from the principles of the invention. A preferred poly(oxyethylene)chitosan according to the present invention has a weight-average molecular weight of about 200 kD to about 600 kD.

In a preferred embodiment, a premix for a hydrogel contains a polybasic carboxylic acid comprising an acidic polysaccharide such as hyaluronan, oxidized hyaluronan, or carboxymethylcellulose. An acidic polysaccharide, hyaluronan bears an ionizable carboxylic acid group on every other monosaccharide residue. The hyaluronan can be in the form of a hyaluronate, that is, with at least most of the carboxylic acid groups being in the ionized or salt form. Sodium hyaluronate is a specific example. A hyaluronan may be of any of a wide range of degrees of polymerization (molecular weights), but a preferred hyaluronan has a molecular weight of about 2,000 kD to about 3,000 kD. Carboxymethylcellulose likewise is an acidic polysaccharide that can be formed semi-synthetically by the reaction of cellulose with sodium chloroacetate. Typically, carboxymethylcellulose has a degree of substitution of 1.0 or less. Oxidized hyaluronan is a polysaccharide in which bonds between vicinal diol units of the sugar moieties of the dextran, a polymer of glucose, have been cleaved and aldehyde groups introduced, but which also retains acidic carboxylic acid groups.

Another preferred embodiment of a premix that forms a hydrogel according to the present invention comprises an acrylated chitosan. A preferred degree of substitution of the chitosan backbone with acrylate groups according to the present invention is about 0.25 to about 0.45. The number of monomeric units that make up a acrylated chitosan according to the present invention may vary widely without departing from the principles of the invention. A preferred acrylated chitosan has a molecular weight of about 200 kD to about 600 kD.

A premix that includes an acrylated chitosan can also include a polybasic carboxylic acid comprising a dicarboxylic acid. One class of dicarboxylic acid that can be used are linear alkyl dicarboxylic acids. The dicarboxylic acid serves to crosslink acrylated chitosan chains through the intermolecular formation of bonds, ionic or covalent, between the chitosan amino groups and the carboxylic acid groups of the dicarboxylic acid. Specific examples of dicarboxylic acids are malonic, succinic, glutaric, adipic, pimelic, suberic, azaleic, and sebacic acid.

A premix that includes an alkylated chitosan can also include a polybasic carboxylic acid comprising carboxymethylcellulose. Carboxymethylcellulose is a derivative of cellulose (a β-1,4 linked polymer of glucose) wherein hydroxyl groups are substituted with carboxymethyl (—CH₂CO₂H) moieties, usually mostly the primary hydroxyl group of each monomeric unit. It is understood that the term carboxymethylcellulose comprises salts of carboxymethylcellulose, such as the sodium salt. An example of a premix comprises acrylated chitosan and carboxymethylcellulose. Carboxymethylcellulose, as is well-known in the art, may have varying degrees of substitution, a “degree of substitution” referring to the number of derivatizing groups, herein carboxymethyl, per each monomer unit on the average. A preferred carboxymethylcellulose according to the present invention has a degree of substitution of about 0.7 and a molecular weight of about 80 kD.
Another embodiment of a premix includes oxidized dextran or oxidized hyaluronan plus an alkylated chitosan. Oxidized dextran is formed semi-synthetically by oxidation of the polysaccharide dextran with a suitable oxidizing agent such as sodium periodate, resulting in the formation of aldehyde groups from the vicinal diols of the glucose monomeric units comprising the dextran. Oxidized hyaluronan is similarly prepared from hyaluronic acid. It is believed that the amino groups of the chitosan derivative react with the aldehyde groups of the oxidized polysaccharide to form imine bonds (“Schiff bases”).

A premix according to the present invention comprises an aqueous medium. An aqueous medium includes water, and may include other components including salts, buffers, co-solvents, additional cross-linking reagents, emulsifiers, dispersants, electrolytes, radiopaque materials, or the like.

A premix according to the present invention may further comprise a dehydrating reagent. A preferred dehydrating reagent is a dehydrating reagent that is sufficiently stable when dissolved or dispersed in an aqueous medium to assist in driving the formation of amide bonds between a chitosan derivative and a polybasic carboxylic acid before it is hydrolyzed by the water in the aqueous medium. A particularly preferred type of dehydrating reagent is a carbodiimide, which is transformed into a urea compound through incorporation of the elements of water. A water-soluble carbodiimide, such as 1-ethyl-3-(N,N-dimethylpropyl)carbodiimide (EDCI), is particularly preferred as it is soluble in the aqueous medium and thus does not require a co-solvent or dispersant to distribute it homogeneously throughout the premix. Other water-soluble carbodiimides are also preferred dehydrating reagents.

A premix according to the present invention may comprise a carboxyl activating reagent. A preferred carboxyl activating reagent is a reagent that serves to activate a carboxyl group towards formation of a new bond by reaction with a nucleophile. A bond such as an amide or ester bond can be formed with an amine or a hydroxyl-bearing compound, respectively. A carboxyl activating reagent can react with the carboxyl group to form a new compound as an intermediate, which then further reacts with another substance such as an amine to form an amide, or a hydroxyl-bearing compound to form an ester. A example of a carboxyl activating reagent is an N-hydroxy compound. An N-hydroxy compound reacts with a carboxyl group to form an N-hydroxy ester of the carboxylic acid, which may subsequently react with, for example, an amino group to form an amide. An example of an N-hydroxy compound is N-hydroxysuccinimide. Another example is N(1)-hydroxybenzotriazole.

Another carboxyl activating reagent is a carbodiimide. A carbodiimide reacts with a carboxyl group to form an O-acylisourea, which may subsequently react with, for example, an amine to form an amide, releasing the carbodiimide transformed through covalent addition of the elements of water to a urea compound. A specific example of carbodiimide is a water-soluble carbodiimide, for example EDCI.

A carbodiimide may serve both as a dehydrating reagent and as a carboxyl activating reagent. Thus, a premix can comprise an alkylated chitosan, a polybasic carboxylic acid, and a carbodiimide. Another preferred embodiment is a premix comprising an alkylated chitosan, a polybasic carboxylic acid, a carbodiimide, and another carboxyl activating reagent. Another preferred embodiment is a premix comprising an alkylated chitosan, a polybasic carboxylic acid, a carbodiimide, and another molecular species wherein that species is a dehydrating reagent.

The substantially liquid premix for a hydrogel comprising the GJI can be directly introduced to the target tissue, for example by using a cannula. After introduction of the premix, gelation results in the hydrogel containing the GJI, which is released over a period of time into surrounding tissue.

Alternatively, the liquid premix can be distributed onto a medical device, as by spraying, for example by electrospraying, or by dipping the device into the premix, or by pumping the premix into voids in the device adapted to receive it, wherein the hydrogel containing the GJI is formed by gelation of the premix. For example, when the medical device is a stent, the stent can be formed of a hollow, porous wire that contains the hydrogel formulation of the invention, which can be emplaced by pumping or pouring the substantially liquid premix into the interior of the hollow, porous wire. Regardless of how the hydrogel formulation of the invention is disposed on the medical device, it is adapted to release the GJI into the surrounding tissue following implantation of the device. The medical device can be implanted surgically in proximity to the ischemic core area in a patient afflicted with stroke or the like; for example, a stent that is coated with the hydrogel containing the GJI may be emplaced within a cerebral artery by introduction into the femoral artery and movement through the circulatory system to the desired location.

In an embodiment according to the present invention, the polymer containing the GJI may be formed into a fiber or a film. For example, in the embodiment where the polymer comprises EVA, a sample comprising EVA and a GJI may be spun or cast into a form of a fiber or a film. The fiber or film can then be emplaced within the tissue of a patient in need thereof for therapeutic purposes. For example, a film can be emplaced directly onto the surface of target tissue in a surgical procedure.

Alternatively, the fiber or film can be disposed on a medical device such as stent. A stent that comprises the formulation of the invention can be formed of any suitable metal, for example, platinum, stainless steel, or nitinol. A stent is preferably formed from a metal that possesses sufficient elasticity that it may be deformed for emplacement within a blood vessel, but expands upon release within the blood vessel to form a substantially tubular structure or framework to support the interior of the blood vessel.

The stent can assume, among other forms, a single helical structure or a double helical structure. The strand may be hollow and have pores, such that the formulation of the invention is disposed inside the strand. An example is a hollow, porous strand within which a hydrogel comprising a GJI is disposed. Alternatively, the formulation of the invention, in the physical form of a fiber, may be disposed within the axis of a finely helical wire that is supercoiled into a larger helical configuration, the fiber being disposed in the central opening of the finely helical wire, that is, the fiber runs in the volume within the coil of the finely helical wire that itself is formed into the larger helical configuration of the intact stent. The fiber has sufficient elasticity or ductility such that when the stent is disposed within a blood vessel and the larger helix forms to support the interior of the blood vessel, the fiber is not substantially ruptured thereby. Alternatively, the fiber may be wrapped around a strand that forms the stent, or may be woven into the stent structure.

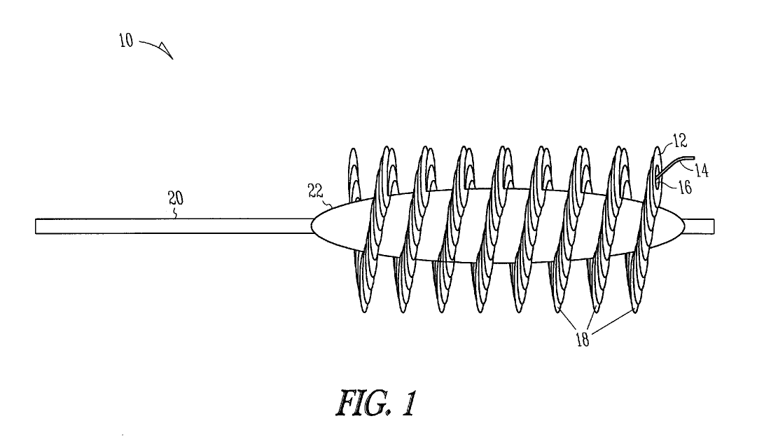

In a preferred embodiment, a thread comprising EVA containing a GJI can be wound or woven around the structure of a stent. For example, referring to FIG. 1, the stent 10 can comprise a finely wound helical wire 12 that can hold the EVA/GJI fiber 14 within the interior opening 16 of the fine helix, which is itself supercoiled into a larger helix 18 forming the stent, such as the stent assumes upon release within a blood vessel. If the stent is of the balloon variety wherein it is expanded in place within the blood vessel by application of gas pressure as through a catheter 20 to a balloon 22 disposed within the stent, the fiber is of sufficient ductility or elasticity to conform to the expanded stent without substantial rupture. Once the stent is emplaced, for example within a cerebral artery such as might be done to reinforce a segment of the artery following removal of an embolus, the medicament-containing polymer thread wound around the stent serves to release the therapeutic GJI into the surrounding tissues.

Alternatively, the GJI/polymer formulation may be dissolved in a suitable solvent, for example dichloromethane in the case of EVA, and the medical device coated with the formulation by dipping or spraying. Following evaporation of the solvent, the device may then be disposed within the tissue, for example a stent within a blood vessel.

The stent can also comprise a pair of wires forming double-helical structure, wherein the two helices are of opposite handedness or are of the same handedness but are 180 degrees out of phase. Here also, a formulation of the invention can comprise a fiber or film that is attached to the stent wires, or can comprise a hydrogel that is disposed in or on the stent wires.

Alternatively, the medical device can be a device other than a stent, for example, biodegradable microspheres adapted for implantation within brain tissue.

### EXAMPLES

### Example 1

### Preparation of Oxidized Dextran

Dextran (5 g) was dissolved in 400 mL of distilled H₂O, then 3.28 g of NaIO₄ dissolved in 100 mL ddH₂O was added. The mixture was stirred at 25° C. for 24 hrs. 10 ml of ethylene glycol was added to neutralize the unreacted periodate following by stirring at room temperature for an additional hour. The final product was dialyzed exhaustively for 3 days against doubly distilled H₂O, then lyophilized to obtain a sample pure oxidized dextran.

### Example 2

### Hydrogel Formation

A 1 mL sample of 2% aqueous oxidized dextran in water solution was mixed with 1 mL of a 2% aqueous acrylated chitosan solution. The mixture was gently stirred for 10 seconds. Gelation occurred within 30 seconds at ambient temperature.

### Example 3

### Analyses of Oxidized Dextran

The degree of oxidation of the oxidized dextran was determined by quantifying the aldehyde groups formed using t-butyl carbazate titration via carbazone formation. A solution of oxidized dextran (10 mg/ml in pH 5.2 acetate buffer) was prepared; and a 5-fold excess tert-butyl carbazate in the same buffer was added and allowed to react for 24 hrs at ambient temperature, then a 5-fold excess of NaBH₃CN was added. After 12 hrs, the reaction product was precipitated three times with acetone and the final precipitate was dialyzed thoroughly against water, followed by lyophilization. The degree of oxidation (i.e., abundance of aldehyde groups) was assessed using ¹H NMR by integrating the peaks: 7.9 ppm (proton attached to tert-butyl) and 4.9 ppm (anomeric proton of dextran).

### Example 4

### Preparation of Oxidized Hyaluronan

Sodium hyaluronan (1.0 gram) was dissolved in 80 ml of water in a flask shaded by aluminum foil and sodium periodate (various amounts) dissolved in 20 ml water was added dropwise to obtain oxidized hyaluronan (oHA) with different oxidation degrees. The reaction mixture was incubated at ambient temperature and 10 ml of ethylene glycol was added to neutralize the unreacted periodate following by stirring at room temperature for an additional hour. The solution containing the oxidized hyaluronan was dialyzed exhaustively for 3 days against water, then lyophilized to obtain pure product (yield: 50-67%).

### Example 5

### Analyses of Oxidized Hyaluronan

The degree of oxidation of oxidized hyaluronan was determined by quantifying aldehyde groups formed with t-butyl carbazate titration via carbazone formation [13]. A solution of the oxidized hyaluronan (10 mg/ml in pH 5.2 acetate buffer) and a 5-fold excess tertbutyl carbazate in the same buffer were allowed to react for 24 hrs at ambient temperature, followed by the addition of a 5-fold excess of NaBH₃CN. After 12 hrs, the reaction product was precipitated three times with acetone and the final precipitate was dialyzed thoroughly against water, followed by lyophilization. The degree of oxidation (i.e., abundance of aldehyde groups) was assessed using ¹H NMR by integrating the peaks: 1.32 ppm (tert-butyl) and 1.9 ppm (CH₃ of hyaluronic acid).

### Example 6

Preparation of Acrylated Chitosan

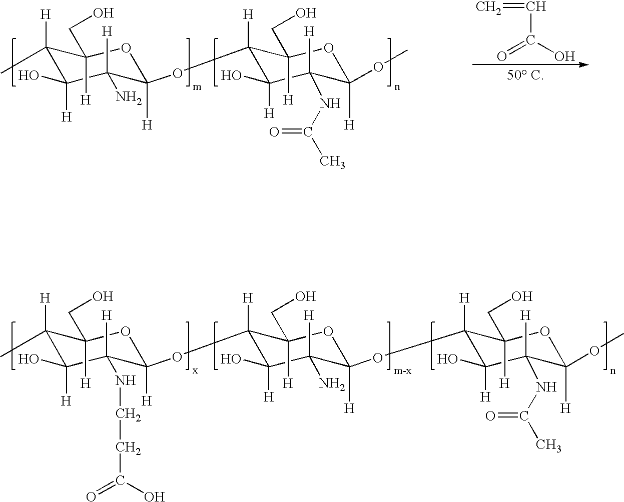

5.52 ml of acrylic acid was dissolved in 150 ml of double distilled water and 3 g of chitosan (Kraeber® 9012-76-4, molecular weight 200-600 kD) was added to it. The mixture was heated to 50 C and vigorously stirred for 3 days. After removal of insoluble fragments by centrifugation, the product was collected and its pH was adjusted to 11 by adding NaOH solution. The mixture was dialyzed extensively to remove impurities.

### Example 7

### Preparation of PEG-Chitosan

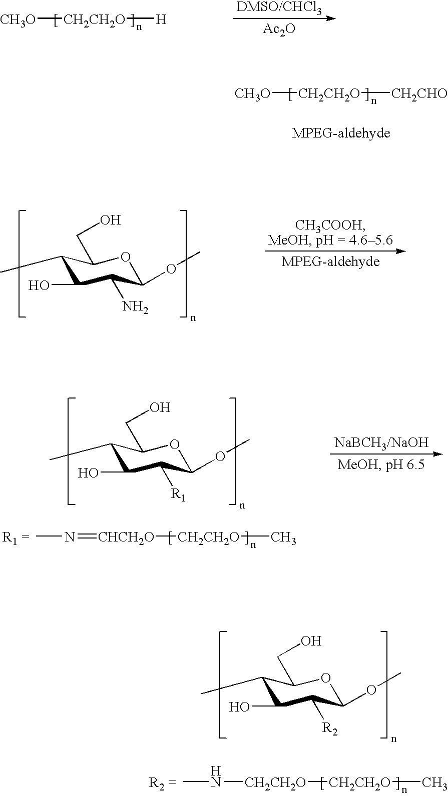

Monomethyl-PEG-aldehyde was prepared by the oxidation of Monomethyl-PEG (MPEG)with DMSO/acetic anhydride: 10 g of the dried MPEG was dissolved in anhydrous DMSO (30 ml) and chloroform (2 ml). Acetic anhydride (5 ml) was introduced into the solution and the mixture is stirred for 9 h at room temperature. The product was precipitated in 500 ml ethyl ether and filtered. Then the product was dissolved in chloroform and re-precipitated in ethyl ether twice and dried.

Chitosan (0.5 g, 3 mmol as monosaccharide residue containing 2.5 mmol amino groups, Kraeber 9012-76-4, molecular weight 200-600 kD) was dissolved in 2% aqueous acetic acid solution (20 ml) and methanol (10 ml). A 15 ml sample of MPEG-aldehyde (8 g, DC: 0.40) in aqueous solution was added into the chitosan solution and stirred for 1 h at room temperature. Then the pH of chitosan/MPEG-monoaldehyde solution was adjusted to 6.0-6.5 with aqueous 1 M NaOH solution and stirred for 2 h at room temperature. NaCNBH₃ (0.476 g, 7.6 mmol) in 7 ml water was added to the reaction mixture dropwise and the solution was stirred for 18 h at room temperature. The mixture was dialyzed with dialysis membrane (COMW 6000-8000) against aqueous 0.5 M NaOH solution and water alternately. When the pH of outer solution reached 7.5, the inner solution was centrifuged at 5,000 rpm for 20 min. The precipitate was removed. The supernatant was freeze-dried and washed with 100 ml acetone to get rid of unreacted MPEG. After vacuum drying, the final product (white powder) was obtained as water soluble or organic solvent soluble PEG-g-Chitosan. The yield of water soluble derivatives was around 90% based on the weight of starting chitosan and PEG-aldehyde.

### Example 8

### Preparation of a Premix of PEG-Chitosan and Hyaluronan

Hyaluronan (sodium hyaluronate, Kraeber 9067-32-7) was dissolved in water as a 0.5% solution by weight. PEG-chitosan, prepared as described in Example 2, was dissolved in water as a 5% solution by weight. A sample of each solution (0.5 mL of each) was mixed, then a solution of EDCI (20 μL of a solution in water at 350 mg/mL) was added and the solution was thoroughly mixed. Immediately a solution of N-hydroxysuccinimide (20 μL of a solution in water at 125 mg/mL) was added and thoroughly mixed in to form a premix. The premix gelled into a hydrogel in about 7 minutes at ambient temperature (22° C.). At 37° C. gelation occurred in about 2 minutes.

### Example 9

### Preparation of a Premix of Acrylated Chitosan and Adipic Acid

A sample of acrylated chitosan prepared as described in Example 1 was dissolved in water at a concentration of 2% by weight. A sample of this solution (0.5 mL) was mixed with a solution of adipic acid in water (40 μL of a 20 mg/mL solution), then a solution of EDCI (20 μL of a 350 mg/mL solution) and the solution thoroughly mixed. Then, a solution of N-hydroxysuccinimide in water (20 μL of a 125 mg/mL solution) was mixed in. The premix gelled in about 9 minutes at ambient temperature (22° C.). At 37° C. gelation occurred in about 3 minutes.

### Example 10

Preparation of a Premix of Acrylated Chitosan and Carboxymethylcellulose

A sample of acrylated chitosan prepared as described in Example 1 was dissolved in water at a concentration of 2% by weight. A sample of carboxymethylcellulose sodium salt (Polysciences no. 06140, MW 80 kD, degree of substitution 0.7) was dissolved in water at a concentration of 5% by weight. These two solutions (0.25 mL each) were mixed with a solution of EDCI (20 μL of a 6.5% solution) and the solution thoroughly mixed. Then, a solution of N-hydroxysuccinimide in water (20 μL of a 35% solution) was mixed in. The solution gelled in about 10 minutes at ambient temperature (22° C.).

In the claims provided herein, the steps specified to be taken in a claimed method or process may be carried out in any order without departing from the principles of the invention, except when a temporal or operational sequence is explicitly defined by claim language. Recitation in a claim to the effect that first a step is performed then several other steps are performed shall be taken to mean that the first step is performed before any of the other steps, but the other steps may be performed in any sequence unless a sequence is further specified within the other steps. For example, claim elements that recite “first A, then B, C, and D, and lastly E” shall be construed to mean step A must be first, step E must be last, but steps B, C, and D may be carried out in any sequence between steps A and E and the process of that sequence will still fall within the four corners of the claim.

Furthermore, in the claims provided herein, specified steps may be carried out concurrently unless explicit claim language requires that they be carried out separately or as parts of different processing operations. For example, a claimed step of doing X and a claimed step of doing Y may be conducted simultaneously within a single operation, and the resulting process will be covered by the claim. Thus, a step of doing X, a step of doing Y, and a step of doing Z may be conducted simultaneously within a single process step, or in two separate process steps, or in three separate process steps, and that process will still fall within the four corners of a claim that recites those three steps.

Similarly, except as explicitly required by claim language, a single substance or component may meet more than a single functional requirement, provided that the single substance fulfills more than one functional requirement as specified by claim language.

It is to be understood that while the invention has been described in conjunction with the detailed description thereof, the foregoing description is intended to illustrate and not limit the scope of the invention, which is defined by the scope of the claims. Other aspects, advantages, and modifications are within the scope of the claims and will doubtless be apparent to persons of ordinary skill in the art.

We claim:

1. A formulation for the treatment or prevention of neural ischemic damage or for treatment of stroke, hemorrhage, trauma, epilepsy, tumor, or any disease of the brain, the formulation comprising a mixture of a substantially solid, substantially water-insoluble, biocompatible, polymeric material, and a gap junction inhibitor.

2. The formulation of claim 1 wherein the gap junction inhibitor is a gap junction inhibitor known to effectively treat or prevent neural ischemic damage.

3. The formulation of claim 1 wherein the gap junction inhibitor is carbenoxolone.

4. The formulation of claim 1 wherein the formulation is biodegradable.

5. The formulation of claim 1 wherein the polymeric material comprises ethylene vinyl acetate copolymer.

6. The formulation of claim 1 wherein the polymeric material comprises poly(methyl methacrylate).

7. The formulation of claim 1 comprising a substantially solid hydrogel.

8. The formulation of claim 7 wherein the hydrogel is formed by gelation of a substantially liquid premix.

9. The formulation of claim 7 wherein the hydrogel comprises chitosan or a chitosan derivative.

10. The formulation of claim 9 wherein the hydrogel further comprises a polybasic carboxylic acid or an oxidized polysaccharide.

11. The formulation of claim 10 wherein the polybasic carboxylic acid comprises an acidic polysaccharide.

12. The formulation of claim 11 wherein the acidic polysaccharide comprises hyaluronan, carboxymethylcellulose, or oxidized hyaluronic acid.

13. The formulation of claim 10 wherein the polybasic carboxylic acid comprises an alkylene dicarboxylic acid of about 3 to about 12 carbon atoms.

14. The formulation of claim 10 wherein the oxidized polysaccharide comprises oxidized dextran or oxidized hyaluronic acid.

15. The formulation of claim 9 wherein the chitosan derivative comprises a poly(oxyalkylene)chitosan or an acrylated chitosan.

16. The formulation of claim 10 further comprising a carboxyl activating reagent and a dehydrating reagent.

17. The formulation of claim 16 wherein the dehydrating reagent is a carbodiimide.

18. The formulation of claim 17 wherein the carbodiimide is EDCI.

19. The formulation of claim 16 wherein the carboxyl activating reagent is an N-hydroxy compound.

20. The formulation of claim 19 wherein the N-hydroxy compound is N-hydroxysuccinimide or N-hydroxybenztriazole.

21. A method for treatment or prevention of neural ischemic damage or for treatment of stroke, hemorrhage, trauma, epilepsy, tumor, or any disease of the brain, comprising implanting the formulation of claim 1 within the target tissue of a patient in need thereof.

22. The method of claim 21 wherein the formulation is implanted by means of a cannula.

23. The method of claim 21 comprising implantation of the formulation in proximity to an ischemic focus.

24. The method of claim 21 wherein the target tissue comprises central nervous system tissue.

25. The method of claim 21 wherein the formulation is disposed in or on a medical device that is adapted for implantation within the target tissue of a patient in need thereof.

26. The method of claim 25 wherein the medical device with the formulation disposed thereon or therein is implanted within the target tissue of a patient in need thereof.

27. The method of claim 25 wherein the medical device is a stent.

28. The method of claim 25 wherein the formulation is a mixture of the gap junction inhibitor and ethylene vinyl acetate copolymer, or poly(methyl methacrylate), or both.

29. The method of claim 28 wherein the formulation is in a physical form of a fiber or a film.

30. The method of claim 28 wherein the formulation is disposed on a stent.

31. The method of claim 29 wherein the fiber is wound around or woven into the stent.

32. The method of claim 29 wherein the stent comprises a finely helical wire supercoiled into a larger helical configuration, and the fiber is disposed in the central opening of the finely helical wire.

33. The method of claim 27 wherein the stent is formed of a hollow, porous strand and a formulation comprising a hydrogel is disposed therein.

34. The method of claim 25 wherein the formulation comprises a hydrogel and the hydrogel is disposed on the medical device by dipping or spraying the device with a premix of the hydrogel or pumping the premix into a void in the device.

35. The method of claim 29 wherein the film is emplaced directly on the tissue of a patient in need thereof.

36. The method of claim 27 wherein the stent is introduced into a patient in need thereof by means of a catheter.

37. The method of claim 27 wherein the stent is formed of stainless steel, nitinol, or platinum.
